(19) United States
(12) Patent Application Publication
(10) Pub. No.: US 2022/0339156 A1
(43) Pub. Date: Oct. 27, 2022

(54) COMPOSITIONS AND METHODS FOR TREATMENT OF DISORDERS ASSOCIATED WITH CLEC16A DYSFUNCTION OR LOSS

(71) Applicant: THE CHILDREN'S HOSPITAL OF PHILADELPHIA, Philadelphia, PA (US)

(72) Inventors: Hakon Hakonarson, Malvern, PA (US); Rahul Pandey, Wallingford, PA (US); Marina Bakay, Parkesburg, PA (US); Heather Hain, Ambler, PA (US)

(73) Assignee: THE CHILDREN'S HOSPITAL OF PHILADELPHIA, Philadelphia, PA (US)

(21) Appl. No.: 17/641,665

(22) PCT Filed: Sep. 9, 2020
(86) PCT No.: PCT/US2020/050015

§ 371 Date: Mar. 9, 2022

Related U.S. Application Data
(60) Provisional application No. 62/897,983, filed on Sep. 9, 2019.

(51) Int. Cl. A61K 31/519; A61K 31/436; A61P 37/02; C12Q 1/6883

(52) U.S. Cl. CPC A61K 31/519; A61K 31/436; A61P 37/02; C12Q 1/6883

(57) ABSTRACT

Compositions and methods for the treatment of CLEC16A associated disorders are disclosed.

This application claims priority to U.S. Provisional Application No. 62/897,983 filed Sep. 9, 2019, the entire contents being incorporated herein as though set forth in full.

### FIELD OF THE INVENTION

The present invention relates the fields of genetic testing and treatment of autoimmune disorders. More specifically, the invention provides new targets and biochemical pathways for screening agents useful for the treatment of autoimmune disorders.

### BACKGROUND OF THE INVENTION

Several publications and patent documents are cited through the specification in order to describe the state of the art to which this invention pertains. Each of these citations is incorporated herein by reference as though set forth in full.

CLEC16A is a well-established autoimmune disorder susceptibility gene and has been associated with several autoimmune diseases, including type-1 diabetes, multiple sclerosis, primary adrenal insufficiency, Crohn's disease, primary biliary cirrhosis, juvenile idiopathic arthritis, rheumatoid arthritis, and alopecia areata, suggesting that CLEC16A could be a master regulator of aberrant autoimmune responses. Despite the strong association of CLEC16A across numerous autoimmune and inflammatory disorders, little is known about CLEC16A's physiological function or its role in disease pathogenesis. Several studies have described the role of CLEC16A in autophagy processes. Previous studies show that loss of CLEC16A leads to an Nrdp1 targeting of Parkin, a master regulator of mitophagy, and that golgi-associated CLEC16A negatively regulates autophagy via modulation of mTOR activity. How this relates to the autoimmune function is yet to be determined.

In our previous work in type-1 diabetes, the protective CLEC16A alleles were associated with higher levels of CLEC16A (formally known as KIAA0350). We also recently showed that ubiquitous loss of CLEC16A led to disrupted mitophagy in immune cells.

Given the large number of autoimmune and other disorders correlated with CLEC16A loss or dysfunction, it is clear that new treatments and therapeutic agents which ameliorate the effects of malfunctioning CLEC16A are urgently needed.

### SUMMARY OF THE INVENTION

The current invention implicates CLEC16A as a critical regulator of lipophagy and endoplastmic reticulum (ER) stress, involving a robust cytokine response that results in a systemic inflammatory response and lipolytic processes responsible for substantive weight loss. As CLEC16A is a major autoimmune gene associated with at least 16 autoimmune diseases, including type-1 diabetes, multiple sclerosis, lupus and inflammatory bowel disease.

In accordance with the present invention, a combination therapy for the treatment of at least one autoimmune disorder associated with aberrant CLEC16A function is provided. In one aspect a method for treating CLEC16A-associated degeneration of the thymus is provided comprising administration of an agent that modulates expression of one or more of CD163, Bcl-2, Pax-5, V-cam1, CD8 and FoxP3, thereby altering the medulla cortex ratio in thymus and ameliorating symptoms associated with degeneration of the thymus. In another embodiment, a method for treating CLEC16A-associated degeneration of spleen is provided comprising administration of an agent that modulates expression of one or more of CD163, CD68, Bcl-2, CD40, Pax5, Vcam1, CD3, and GzmB, thereby altering the white red pulp ratio in the spleen and ameliorating symptoms associated with degeneration of the spleen. The invention also discloses a method for treating CLEC16A-associated degeneration of the pancreas comprising administration of an agent which modulates CD163 expression and/or immune cell infiltration and/or acini cell degeneration in the pancreas, thereby reducing autoimmune symptoms.

As noted above, the invention includes a combination the combination therapy that can include at least two of a mitophagy suppressor/modulator, ER suppressor, a JAK2 inhibitor and SOCS1 inhibitor. Inhibitors of the JAK-Stat pathway will also have utility in the present invention. In certain embodiments, the agents are rapamycin and tofacitinib. In other embodiments, a nucleic acid encoding CLEC16A is obtained from the subject and assessed for the presence of genetic alterations.

### BRIEF DESCRIPTION OF THE DRAWINGS

FIG. 1. Percent body weight from initial body weight over time. Mice were weighted 3 times per week during the study.

FIG. 2. Clec16a KO male and female mice exhibit complete loss of body and visceral fat.

FIG. 3. Foot intake in KO mice as compared to controls.

FIGS. 4A-4D. FIG. 4A) Representative immunoblot depicting CLEC16A expression. FIG. 4B) mRNA levels of ER stress markers in control and KO gWAT. FIG. 4C) Representative immunoblot depicting ER stress in KO gWAT. FIG. 4) Organ weight ratio.

FIG. 5. Hormone sensitive lipase (HSL) phosphorylation in control and KO mice.

FIGS. 6A-6C. FIG. 6A) The mRNA expression and Immunoblot (FIGS. 6B and 6C) of lipid catabolism genes (Cpt1b, Ppara), adipogenic genes (Pparg and Adipoq) and thermogenic genes (Ucp1 and Cidea) from gWAT of control and KO mice.

FIG. 7. Lipid analysis (cholesterol, triglyceride, and free fatty acid) from Clec16a KO vs. control mouse serum.

FIGS. 8A-8D. FIG. 8A) Clec16a KO mice exhibit decreased adiponectin, leptin, and LDL-R in comparison to control. FIG. 8B) Clec16a KO mice exhibit up increased cytokines, chemokines, growth factors compared to control adipose. FIG. 8C) Clec16a KO mice exhibit up increased cytokines, chemokines, growth factors compared to control adipose. Inhibitor U0126 reverses the upregulation. FIG. 8D) High constitutive expression of IL-16 precursor in Clec16a KO splenocytes and release of bioactive IL-16 by active caspase-3.

FIG. 9. Decreased expression of SOCS1 and SOCS3 in Clec16a KO splenocytes.

FIGS. 10A-10I. Tofacitinib, pan JAK/STAT inhibitor, partially rescues the lipodystrophic phenotype and improves survival of UBC-Cre-Clec16aloxP KO mice. SOCS1 (FIG. 10G) and SOCS3 (FIG. 10H) expression by RT-PCR in control, KO±tofacitinib mice. (FIG. 10I) Predominant Th-1 Cytokine/chemokine in Clec16a KO and rescue with Tofacitinib. The representative graph is quantification of cytokines and chemokine from plasma of Control (Vehicle), KO and KO+Tofacitinib inhibitor treated mice using the Mouse Cytokine Array panel.

FIG. 11. Rapamycin attenuates the lipodystrophic phenotype and improves survival KO mice.

FIG. 12. ANA-9 line Immunoblot assay. Lane 1 positive control is showing all the antigens. Lane 2 &3 are probed with sera from control mice; lane 4-8 are probed with sera of Clec16a KO mice.

FIG. 13. Serum Immunoglobulin Isotyping. ELISA was performed to evaluate changes in serum Immunoglobulins isotypes, isotypes and IgG subclasses with control mice and KO mice sera (n=10).

FIG. 14. Clec16a knockout induces disability in mice. KO mice exhibit abnormal neurons in the dorsal root ganglia due to dysregulated mitophagy. Activated microglia with inflammation in the spinal cord dorsal columns and loss of cerebellar Purkinje cells is evident.

FIG. 15. Clec16a KO DRG, TG exhibit dysregulated ER stress and OXPHOS signaling depicted by RT-PCR and Immunoblot analysis.

FIGS. 16A-16E. (FIG. 16A) CLEC16A Immunoprecipitation pull down and blot. (FIG. 16B) Gel image for MSMS. (FIG. 16C) Top ten predicted candidate partners interacting with Clec16a. (FIG. 16D) Immunoblot blot depicting constitutively elevated ISG15 in neurological tissues. (FIG. 16E) Model depicting modification of proteins through ISG15 referred as ISGlyation. USP43 mediates the reversal.

FIGS. 17A-17B. (FIG. 17A) Comparison of red and white pulp ratios in control and CLEC16A KO mice. (FIG. 17B) Graphs quantifying the changes in the spleen of KO and wild type mice.

FIGS. 18A-18B. (FIG. 18A) Comparison of cortex and medulla ratios in control and CLEC16A KO mice. (FIG. 17B) Graphs quantifying the changes in the thymus of KO and wild type mice.

FIG. 19. Immunohistochemistry showing the changes in immune cell infiltration and acini cell degeneration in wild type and CLEC16A KO mice.

FIG. 20. A graph showing relative fold change in a variety of cellular markers between control and KO mice at Day 23 post-induction of Clec16a KO.

FIG. 21. Graphs showing significant upregulation of CD163 in thymus and upregulation of CD163 and CD68 in spleen.

FIG. 22 shows graphs depicting upregulation of Bcl-2 in thymus during the course of induction while Bcl-2 was elevated at day 18.

FIG. 23 shows graphs quantifying expression changes in CD40 in thymus and spleen during KO induction.

FIG. 24 shows graphs quantifying expression changes in Pax5, and CD19 in thymus and spleen during KO induction.

FIG. 25 shows graphs quantifying expression changes in Icam1, and Vcam1 in thymus and spleen during KO induction.

FIG. 26 shows graphs quantifying expression changes in CD3 in thymus and spleen during KO induction.

FIG. 27 shows graphs quantifying expression changes in CD8, and GzmB in thymus and spleen during KO induction.

FIG. 28 is a graph quantifying expression changes in FoxP3 in thymus during KO induction.

### DETAILED DESCRIPTION OF THE INVENTION

Recent studies revealed that CLEC16A is an important regulator of autophagy/mitophagy. Given its association with several autoimmune disorders, we generated an inducible global knockout (KO), Clec16a^{ΔUBC} mice to investigate its role in autoimmunity. KO mice exhibited immune dysfunction, severe weight loss through lipolysis, and a neuronal phenotype with an increase in severity over time leading to morbidity. As observed previously, attenuation of CLEC16A disrupted autophagy, mitophagy and caused neurodegeneration. Accelerated mitophagy in cerebellum, cortex, trigeminal ganglia, dorsal root ganglia, and spinal cord was observed in KO mice as compared to control mice.

ER stress and mitochondrial dysfunction results in increased oxidative stress and production of multiple proinflammatory mediators. Dysregulated OXPHOS signaling was observed in DRG's and splenic lysates of the Clec16a^{ΔUBC} KO mice. KO mice also exhibited an inflammatory cytokine/chemokine profile. KO displayed elevated antibody levels, including IgM, IgA, Ig2b, IgG3 and autoantibodies in sera.

While treatment with a JAK/STAT inhibitor (tofacitinib) partially rescued the lipodystrophic phenotype and improved survival, it only rescued the autophagy in neuronal tissues and did not alter the neuronal phenotype. STAT proteins in neuronal tissues showed no significant change with tofacitinib-treatment as compared to untreated Clec16a^{ΔUBC} KO mice. This may be due to the negative feedback loop associated with JAK/STAT/SOC1 signaling.

Our data indicate loss of function variants in CLEC16A that are associated with decreased CLEC16A levels may contribute to autoimmunity through elevated ER Stress resulting in dysregulated mitophagy and autophagy, which contributes to adipose lipolysis and production of inflammatory mediators. Drugs modulating ER stress, mitophagy/autophagy or the JAK/STAT pathway partially reverse the process and may be effective in treating and preventing symptoms of autoimmune disorders in individuals with risk associated CLEC16A variants. A combination of such drugs may be optimal for this.

Clec16a KO mice exhibited increased food consumption and without evidence of hyperglycemia, loss of adipose tissue, severe weight loss, elevated Immunoglobulins and significantly reduced circulating insulin levels. Metabolic analysis revealed disturbances in lipid profile measures. White adipose tissue decreased concomitantly with enhanced inflammatory response and energy wasting. The loss of CLEC16A leads to a vicious cycle of autophagic impairment and endoplasmic reticulum (ER) stress, which contributes to excessive lipolysis and lipotoxicity resulting in activation of JAK/STAT, mTOR, P38 and JNK and release of multiple proinflammatory mediators under compromised mitophagy environment. ER stress is known to activate lipolytic cascade. Aberrant or excess cytokine production plays a key role in driving autoimmune and autoinflammatory disorders. Collectively, our data indicate that loss of CLEC16A induces ER stress, dysregulated autophagy and mitophagy, which in turn activates lipolytic cascade resulting in excessive adipocyte lipolysis which generates lipid mediators and triggers inflammation via activation of JNK/NF-kB signaling pathway.

Thus, CLEC16A exerts its effect on a wide variety of immune cells through modulation of ER stress, SOCS expression and regulation of cytokine signaling, suggesting that perturbations in the molecular link between CLEC16A, ER stress, mitophagy, lipophagy, and SOCS1 may underlie inflammatory and autoimmune disorders. In patients with autoimmune disease, harboring variants that result in CLEC16A hypofunction, drugs with modulatory effects on ER stress/mitophagy/autophagy/SOCS1-JAK-STAT signaling could compensate for the attenuated CLEC16A activity and be developed for targeted interventions. Our Clec16a KO highlights multifaceted roles of Clec16a in normal physiology, including a novel target for weight regulation, as well as mutation-mediated pathophysiology.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

“CLEC16A associated immune disorders include, without limitation, type-1 diabetes, multiple sclerosis, primary adrenal insufficiency, Crohn's disease, primary biliary cirrhosis, juvenile idiopathic arthritis, rheumatoid arthritis, and alopecia areata, uveitis, and lupus.

“Sample” or “patient sample” or “biological sample” generally refers to a sample which may be tested for a particular molecule, preferably an ADHD specific marker molecule, such as a marker described hereinbelow. Samples may include but are not limited to cells, body fluids, including blood, serum, plasma, cerebral spinal fluid, urine, saliva, tears, pleural fluid and the like.

The terms “agent” and “compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the CNV or SNP-containing nucleic acids described herein or their encoded proteins. Agents and compounds may also be referred to as “test agents” or “test compounds” which are evaluated for potential biological activity by inclusion in screening assays described herein below.

Inhibitors of the JAK pathway also have utility in the present invention. These include without limitation those set forth below.

| | |
|---|---|
| Ruxolitinib (INCB18424) | JAK1/JAK2 > TYK2 |
| Baricitinib (INCB28050) | JAK1/JAK2 |
| Peficitinib | Pan-JAK (some selectivity for JAK3) |
| Decemotiniba | JAK3 |
| Filgotinib | JAK1 |
| Solcitinibb | JAK1 |
| Itacitinib (INCB039110) | JAK1 |
| SHR0302 | JAK1 > JAK2, JAK3 |
| Upadacitinib | JAK1 |
| PF-04965842 | JAK1 |

The term “modulate” as used herein refers to increasing/promoting or decreasing/inhibiting a particular cellular, biological or signaling function associated with the normal activities of CLEC16A molecules described herein. For example, the term modulate refers to the ability of a test compound or test agent to interfere with signaling or activity, or promote the activity of a gene or protein of the present invention.

### V. Pharmaceutical and Peptide Therapies

The elucidation of the role played by the CLEC16A described herein in inflammatory signaling facilitates the development of pharmaceutical compositions useful for treatment and diagnosis of autoimmune disorders associated with aberrant CLEC16A function. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As discussed above methods for treating at least one autoimmune disorder associated with aberrant CLEC16A function are provided. In certain embodiments, the combination includes at least two of a mitophagy suppressor/modulator, ER suppressor, a JAK2 inhibitor and SOCS1 inhibitor. Inhibitors of the JAK-Stat pathway will also have utility in the present invention. Such inhibitors are known in the art and include siRNA molecules, peptide mimetics and small molecules such as those listed hereinabove.

The following examples are provided to illustrate certain embodiments of the invention. They are not intended to limit the invention in any way.

### Example I

### Role of CLEC16A and SOCS1 as Therapeutic Targets in Autoimmunity Modelled Through a UBC-Cre-Clec16aloxP Phenotype Mouse

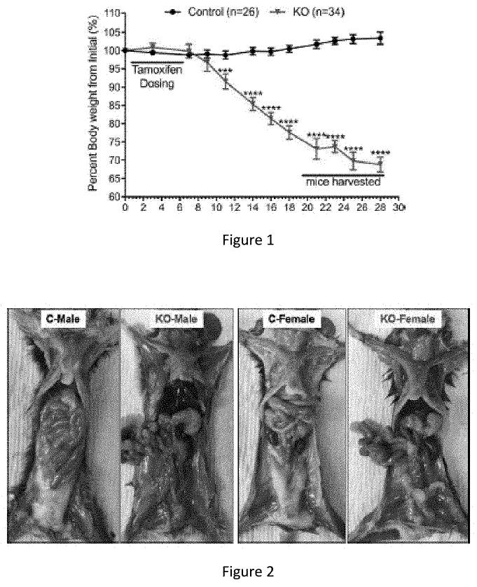

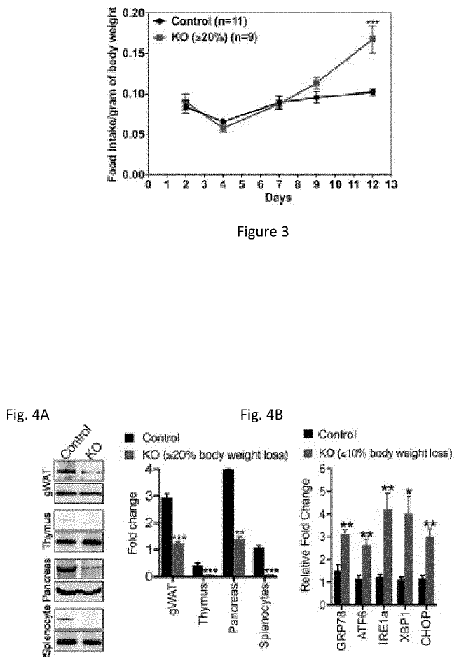

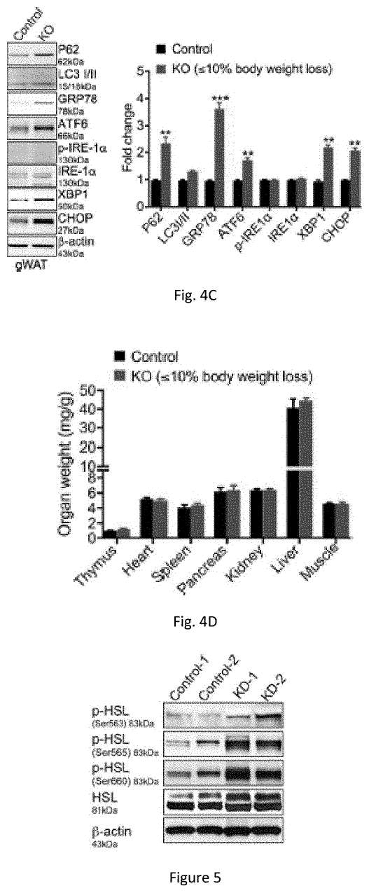

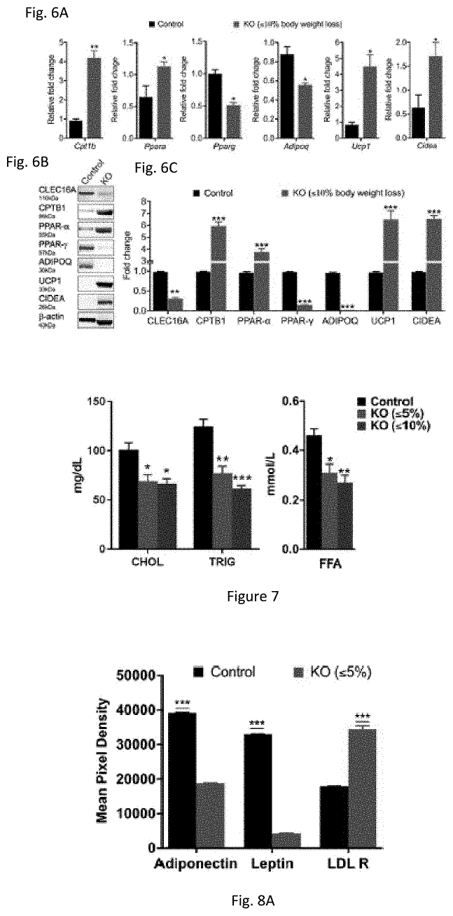

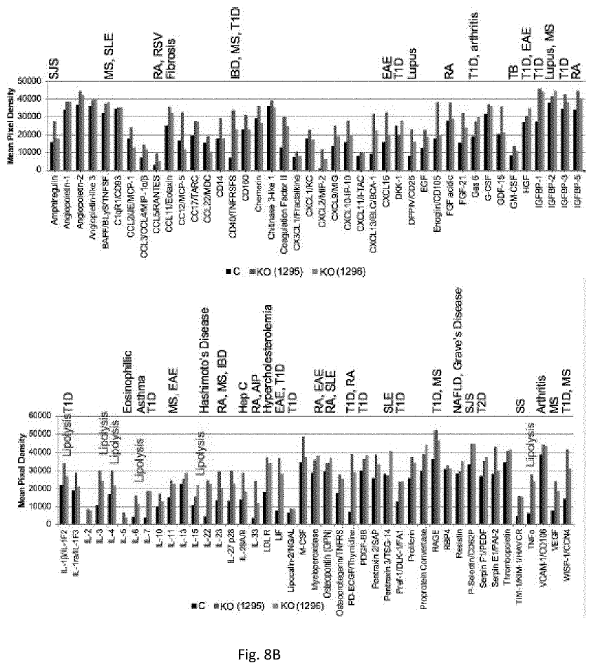

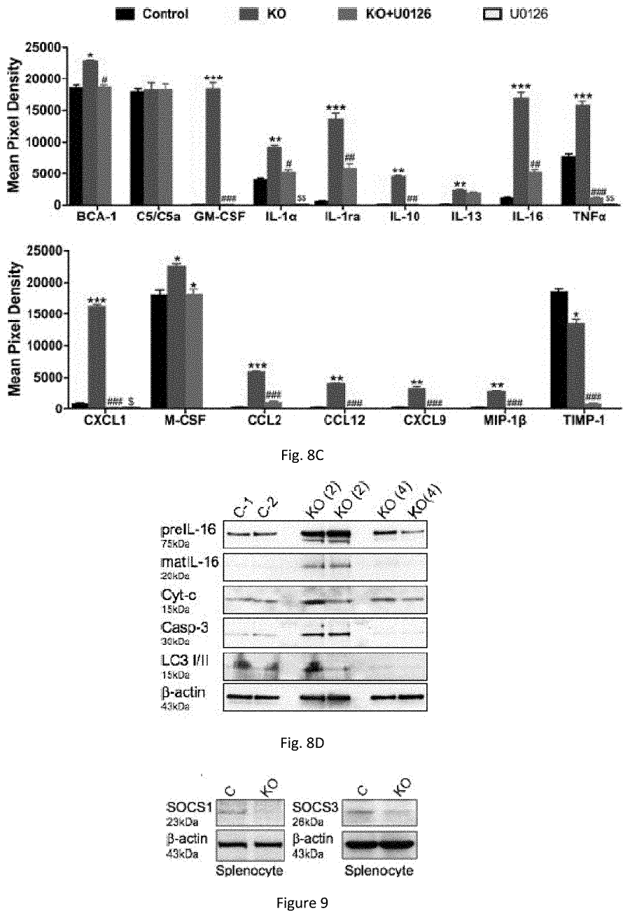

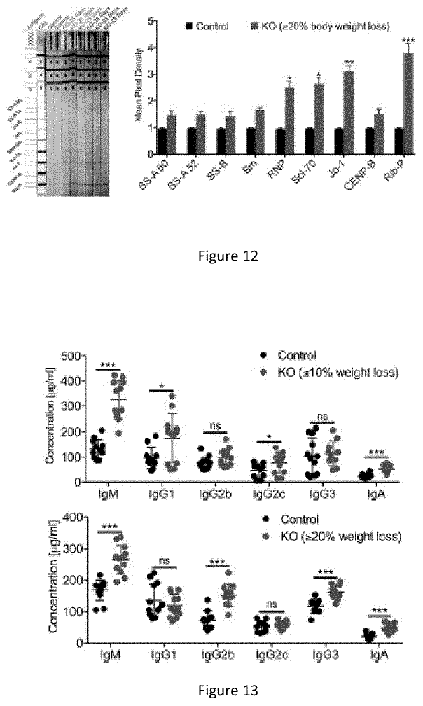

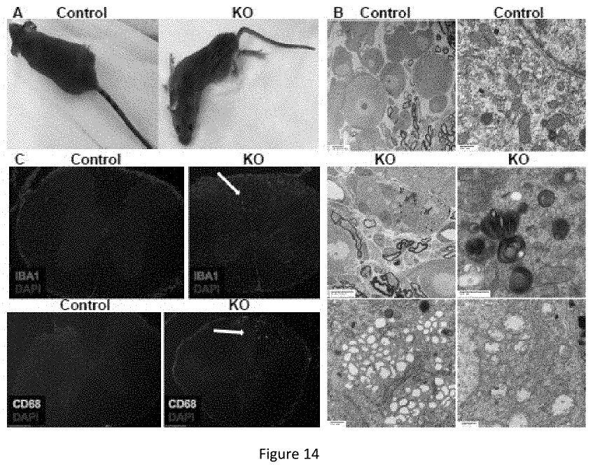

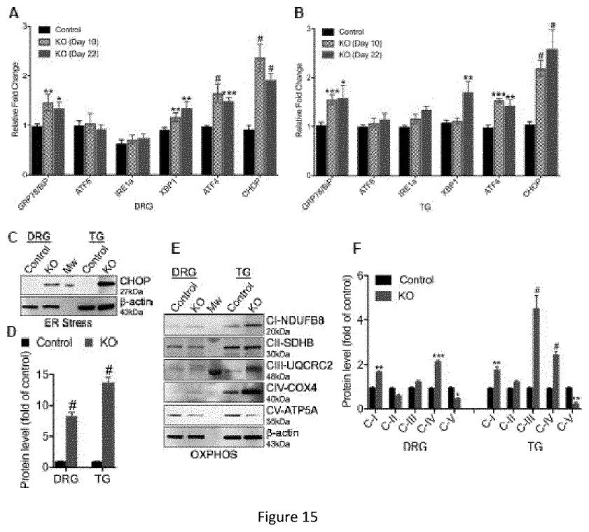

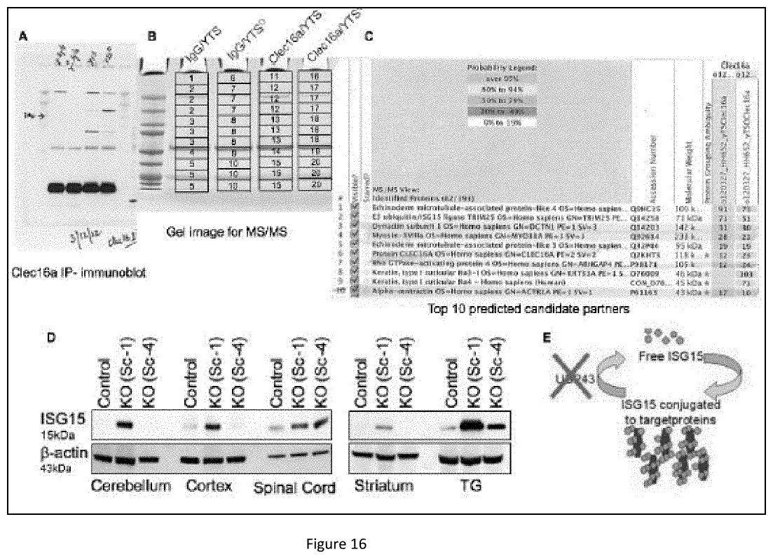

Our Clec16a KO mouse model shows mitochondrial defect and accumulation of unhealthy mitochondria. Our lab and others have shown a connection between Clec16a and autophagy in immunological and neurological cells (Redmann et al., 2016; Soleimanpour et al., 2014; Tam et al., 2017). The first visible observation we made in control and Clec16a KO mice, fed on regular chow diet was difference in body weight. Clec16a knockout mice exhibit significant weight loss and adipose atrophy starting 1 week after initiation of tamoxifen treatment in comparison to control mice (FIGS. 1,2). Increased food intake observed in KO mice fails to rescue the weight loss and adipose tissue atrophy (FIG. 3). Extended work in our laboratory shows that reduced expression of CLEC16A leads to dysregulation of ER homeostasis in adipose and elicits a lipolytic cascade in response to inhibition of autophagy (FIG. 4). We show that the extreme weight loss observed in the Clec16a KO mice is due to lipolysis (lipophagy) observed by the complete loss of fat and an increased phosphorylation of Hormone sensitive lipase (HSL) proteins in Western blots (FIG. 5). mRNA expression and immunoblot analysis revealed upregulation of catabolic and thermogenic genes together with downregulation of downstream adipogenic genes promoting HSL-mediated lipolysis in adipose tissue (FIG. 6). Serum lipid analysis revealed significant decrease in Cholesterol, Triglycerides and free fatty acids (FIG. 7) and decreased Adiponectin, Leptin and upregulated LDL-Receptors in adipose tissue (FIG. 8A). Normal adipose tissue growth and function is critical to maintaining metabolic homeostasis and its excess (e.g. obesity) or absence (e.g. lipodystrophy) is associated with severe metabolic disease. In addition, elevated cytokine levels as measured by Proteome Profiler Mouse XL Cytokine Array were observed concurrent with the lipolysis and could contribute to further wasting and the progressive neurodegeneration resembling spinocerebellar ataxia observed in the mice (FIG. 8). Evidence for a link to SOCS proteins and the JAK/STAT pathway (FIGS. 9,10) with autoimmune inflammatory phenotype (FIGS. 12,13) and spinocerebral ataxia (FIG. 14), elevated ER stress in neuronal tissues (FIG. 15), dysregulated OXPHOS signaling (FIG. 15) are presented respectively. Clec16a mediates its pathogenic effect in certain cases through ISG15, identified by MSMS using Clec16a overexpression NK cell line (FIG. 16). Our whole body inducible Clec16a KO mouse, therefore provides a comprehensive murine model for use in future elucidation of mechanism and dug targets involved in healthy form of weight loss, Autoimmune-inflammatory phenotype and spinocerebellar degeneration.

### Clec16a KO Mice Exhibit Severe Weight Loss.

We employed UBC-Cre-Clec16aloxP mice—an inducible KO model to study CLEC16A's role in autoimmunity. We choose this model to circumvent possible embryonic lethality and determine the effect of CLEC16A loss in adult mice. The first visible observation we made in control and Clec16a KO mice, fed on regular chow diet was difference in body weight. Clec16a knockout mice exhibit severe weight loss starting 1 week after initiation of tamoxifen treatment in comparison to control mice. During the same time period control mice showed a healthy appearance and maintained their body weight throughout the study in comparison to Clec16a KO (FIG. 1).

### Clec16a KO Mice Exhibit Adipose Tissue Atrophy.

All Clec16a KO exhibited significant reduction of body weight which deteriorated over the length of the study with increased severity in neurological symptoms, and an elevated morbidity rate (FIG. 1). According to literature gender differences do exist for T1D pathogenesis, MS and other autoimmune disorders. The disease incidence lays around 60-80% in females and 20-30% in males. For the purpose of healthy examination and to determine the cause of weight loss, the Clec16a KO and control mice were dissected. Compared to control mice, both male and female Clec16a KO mice depicted near to complete absence of typical gonadal adipose fat tissue (FIG. 2). Further examination indicated that all the WAT depots, including gonadal, inguinal, mesenteric, retroperitoneal, perineal, and pericardial, were remarkably reduced or absent in Clec16a KO mice (FIG. 2). Thus, Clec16a loss promotes body weight reduction and fat loss leading to lipodystrophy of adipose tissue, a phenotype similar to that observed in mammalian models with lipodystrophy and has profound effect on WAT deposits in adult mice. These results suggest that Clec16a KO modulates lipid metabolism and triggers abnormal fat loss possibly mediated by lipolysis (lipophagy).

### UBC-Cre-Clec16aloxP KO Mice Exhibit Increased Food Intake.

We performed food intake study to rule out the amount of food consumed as a possible reason behind the severe weight loss. Clec16a KO mice consumed as much or more food in comparison to the control mice. Thus, less food consumption is not the reason behind the weight loss of Clec16a KO mice (FIG. 3). Despite increased food consumption Clec16a KO mice kept losing weight rapidly. These results suggest that CLEC16A modulates fat loss either by reduced efficiency in storage or an increased energy expenditure, or both in the CLEC16A KO mice.

### ER Stress Contributes to Adipose Tissue Atrophy in CLEC16A Knockout Mouse.

We confirmed the reduced expression of CLEC16A protein in gonadal white adipose tissue gWAT, splenocytes, thymus and pancreas from TAM treated (KO) mice by Western blot (FIG. 4A). We hypothesized that reduced expression of CLEC16A leads to dysregulation of ER homeostasis in adipose tissue and elicits the lipolytic cascade in response to inhibition of autophagy. To study if CLEC16A KO evoked ER stress, we first examined gWAT for ER stress by RT-PCR (FIG. 4B) and immunoblot analysis (FIG. 4C). As expected, the ER stress marker genes (GRP78, ATF6, IRE1a, XBP1 and CHOP) showed significant upregulation at mRNA levels in the KO mice (FIG. 4B). Immunoblot analysis further confirmed the above findings. gWAT lysates of KO mice showed significant upregulation GRP78, ATF6, XBP1 and CHOP with barely detectable phospho-IRE1a (FIG. 4C). We also checked the expression of the autophagosome marker LC3-I/II and P62 in gWAT lysate. Clec16a KO gWAT showed significant increase and accumulation of P62 and modest increase in LC3-II expression in comparison to control. Our results show that fat loss had no adverse effect on organ weight in control and KO mice with <10% body weight loss (FIG. 4D). Thus, Clec16a loss in adipose promotes ER stress in response to defective autophagic flux. ER stress possibly activates lipolytic cascade downstream resulting in body weight reduction through adipose tissue inflammation leading to severe generalized lipodystrophy and a profound effect on all WAT deposits.

### Clec16a KO Leads to Abnormal Fat Loss by Accelerated HSL Mediated Lipolysis.

To examine the underlying fat loss in CLEC16A KO, we used immunoblot analysis to assess the role of CLEC16A in inducing lipolysis (lipophagy). HSL (hormone-sensitive lipase) is a key enzyme in the mobilization of fatty acids in adipocytes as well as non-adipocytes. Triacylglycerol is stored in lipid droplets as a primary energy reserve. During lipolysis, triacylglycerols in adipocytes are hydrolyzed into free fatty acids and glycerol. Phosphorylation of HSL at Ser563, Ser659, and Ser660 by PKA stimulates HSL activity, which in turn catalyzes the hydrolysis of triacylglycerol. We found increased phosphorylation of HSL indicating beta-3-adreoreceptors (β3-AR) induced lipolysis (FIG. 5). Our results indicate that CLEC16A functions to restrain lipolysis in adipose tissues and loss of CLEC16A triggers lipolysis.

To gain insight into the mechanism(s) whereby CLEC16A mediates its effect n energy expenditure to induce severe fat and weight loss, we measured expression of key genes regulating lipid metabolism in gWAT of Clec16a KO mice (≤10% body weight loss) by RT-PCR. Virtually no fat was left in mice exhibiting ≥20% body weight loss to analyze. We found that carnitine palmitoyltransferase 1b (Cpt1b), a gene essential for adipose tissue fatty acid oxidation, was significantly upregulated in CLEC16A KO gWAT, along with the upstream transcription factor, peroxisome proliferator-activated receptor alpha (Pparα). Further, the expression of the adipogenic gene, Ppar-α and its downstream target, adiponectin precursor (Adipoq), were significantly reduced in Clec16a KO gWAT. Thermogenic genes, including thermogenin (Ucp1), and the cell death-inducing DFFA-like effector A (Cidea) gene were significantly upregulated in WAT of Clec16a KO mice (FIG. 6A). Importantly, the protein expression also correlated with upregulated mRNA expression in gWAT of Clec16a KO mice. Immunoblot analysis showed significant upregulation in expression of CPTB1, PPAR-α, UCP-1, CIDEA and significant downregulation in expression of PPAR-y and ADIPOQ (FIGS. 6B, C). Thus, fat loss in Clec16a KO mouse is mediated through upregulation of catabolic and thermogenic genes together with downregulation of adipogenic genes promoting HSL-mediated lipolysis. Increased food intake in KO mice fails to rescue the white adipose atrophy.

Lipolysis is defined as the catabolism of triacylglycerols stored in cellular lipid droplets. New findings that lipolytic products and intermediates participate in cellular signaling processes and is particularly important in many non-adipose tissues unveils a previously underappreciated aspect of lipolysis, which may be relevant for human diseases. Normal adipose tissue growth and function is critical to maintaining metabolic homeostasis and its excess (e.g. obesity) or absence (e.g. lipodystrophy) is associated with severe metabolic disease. Decreased triglyceride storage leads to adipocyte lipotoxicity, mitochondrial dysfunction and increased oxidative stress. This contributes to impaired insulin sensitivity and adverted liver, muscles and heart functions leading to early complications.

### Serum Lipid Analysis of Clec16a KO and Control Mice.

For comparison, we split KO mice in two groups, Clec16a KO mice with ≤10% body weight loss and Clec16a KO mice with ≥20% body weight loss, as virtually no fat was left in mice in the latter group. We performed lipid analysis on serum of control and Clec16a KO mice. A significant decrease in cholesterol, triglycerides and free fatty acid was observed in the serum of Clec16a KO mice compared to control (FIG. 6). These results suggest that, controlled Clec16a expression could be of therapeutic potential in promoting a healthier form of weight loss.

### KO Mice Exhibit Increased Cytokine Levels in Adipose Tissue and Plasma.

To gain insight how Clec16a KO, weight loss and lipolysis promote a dynamic immune response in murine adipose tissue and may contribute to disease pathogenesis, we evaluated adipose tissue and plasma of control and Clec16a KO mice in the Proteome Profiler Mouse XL Cytokine Array. The Proteome Profiler Mouse XL Cytokine Array Kit is a membrane-based sandwich immunoassay allows parallel determination of the relative levels of selected mouse cytokines and chemokines. Adiponectin and leptin from adipose tissue play a key role in energy homeostasis and metabolism. Clec16a KO mice exhibit decreased adiponectin, leptin and LDL-R compared to control (FIG. 8A). In addition, we saw up-regulation of several cytokine, chemokines and inflammatory markers in adipose tissue of Clec16a KO mice that have been associated with several autoimmune disorders (FIGS. 8B, C). Our results indicate that the signal originates from dysregulated lipolysis (adipose tissue) and promotes wasting.

To gain insight in the inflammatory mechanism involved in the development, progression and pathogenesis of various autoimmune diseases, we profiled plasma from control, KO and U0126-treated KO mice for cytokines and chemokine using Mouse Cytokine Array panel. Plasma from Clec16a KO mice showed upregulation of Th1 cytokines (TNF-α, IL-1, & IL-16), vs. low levels of Th2 (IL-10 & IL-13) and elevated levels key chemokines GM-CSF, KC (CXCL1) JE (MCP-1), MCP-5, MIG (CXCL9), MIP-1b (CCL4) in comparison to control (FIG. 8D).

U0126 inhibitor treatment reversed all the up regulated cytokines and chemokines, suggesting that the inflammatory mechanism involved with autoimmune risk is mediated by dysregulated mitophagy and can be corrected by mitophagy inhibitors (FIG. 8C). Our results provide critical evidence in support for role of dysregulated lipolysis and Clec16a loss leads to progression of autoimmunity as depicted in graph (FIGS. 8B-C). Increase in IL-16 in the plasma cytokine levels could contribute to the neurological degeneration seen in the Clec16a KO mice (FIG. 8C). Our Immunoblot analysis indicates constitutively high expression of IL-16 precursor and bioactive IL-16 in Clec16a KO splenocyte further support the role of cytokine mediated neurodegeneration (FIG. 8D). The cytokine IL-16 is a CD4+ T cell-specific chemoattractant that is biased towards CD4+ Th1 cells. IL-16 precursor is constitutively expressed in lymphocytes and during CD4+ T cell activation; active caspase-3 cleaves and releases C-terminal bioactive IL-16. The connection between increased cytokines and neurodegeneration is known (Khaibullin et al., 2017). It is also known that the presence of CLEC16A MS risk alleles correlate with reduced SOCS1 and DE DEXI expression in the thymus through a regulatory element (Leikfoss et al., 2013).

### SOCS Protein Expression is Decreased in UBC-Cre-Clec16aloxP KO Mice.

Based on the observed loss of visceral and subcutaneous fat, and food intake study, our ubiquitous Clec16a KO mice display a phenotype similar to that observed in lipodystrophy. Dysregulated lipolysis contributes to lipotoxicity, mitochondrial dysfunction and increased oxidative stress resulting in production of inflammatory mediators. CLEC16's genomic location next to the suppressor of cytokine signaling 1 (SOCS1) gene and the expression specificity in immune cells including dendritic cells, B & T-lymphocytes and natural killer (NK) cells, which are pivotal in the pathogenesis of several autoimmune disorders, led us to hypothesize that CLEC16A exerts its effect on a wide variety of immune cells via modulating SOCS expression and regulating JAK-STAT mediated cytokine signaling. The SOCS (suppressor or cytokine signaling) family members are negative regulators of cytokine signal transduction that inhibit the Jak/Stat pathway. These proteins are important regulators of cytokine signaling, proliferation, differentiation, and immune responses and are involved in regulating over 30 cytokines, including interleukins, growth hormone (GH), interferon, leptin, and leukemia inhibitory factor. SOCS1 shares the most homology with SOCS3 and both are highly induced by cytokines. Both SOCS1 and SOCS3 directly inhibit Jak activity. Jak (Janus Kinase) and Stat (signal transducer and activator of transcription) proteins are play important roles in inflammatory immune responses (Fenner et al., 2006), and therefore, regulation of Jak/Stat signaling is crucial to prevent aberrant signaling which can lead to disease progression.

To examine the mechanism involved in the inflammatory cytokine storm, we examined the levels of SOCS1 and SOCS3 expression in an immunoblot analysis. Splenocytes from Clec16a KO exhibit decreased expression of SOCS1 and SOCS3 compared to control (FIG. 9). With SOCS protein levels decreased, cytokine production is not suppressed resulting in the increased cytokine levels contributing to inflammation, concomitant lipolysis and neurodegeneration observed in the Clec16a KO mice. In light of our data and given the association of Clec16a with several autoimmune disorders, we hypothesize that the molecular link between CLEC16A, lipophagy, and SOCS is abnormal and leads to autoimmune disorders.

### The Pan JAK Inhibitor Tofacitinib Suppresses SOCS1-JAK-STAT Mediated Cytokine Signaling and Improves Survival of Clec16a KO Mice.

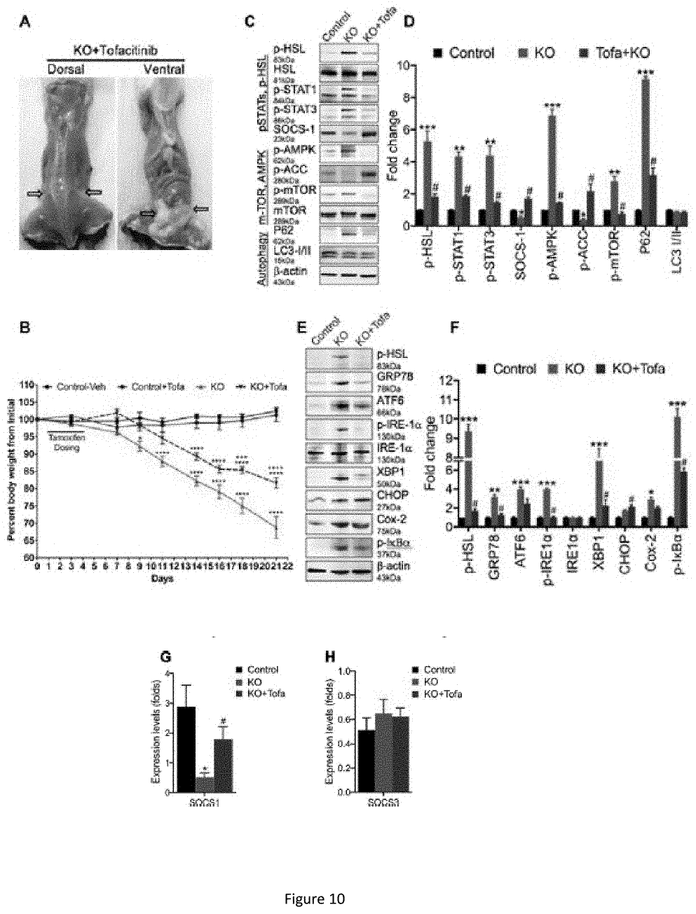

In light of the above findings and established CLEC16A association with several autoimmune disorders, we hypothesized that upregulated JAK/STAT signaling observed in Clec16a KO mice could be rescued using a JAK/STAT inhibitor. Recent discoveries support the emerging view that autoinflammatory diseases may be due to pathological derangement of stress sensing pathways that normally function in host defense. Endoplasmic reticulum (ER)/Unfolded protein response (UPR) is well situated to sense danger and contribute to immune response. Cytokines critically mediate host defense against pathogens, but when aberrantly produced may also drive pathogenic inflammation. We treated Clec16a KO mice with tofacitinib to repurpose the FDA-approved drug for novel clinical indications in future human studies. CLEC16's genomic location next to the SOCS1 gene, and CLEC16's expression specificity in immune cells, makes it an ideal candidate to be explored as potential druggable target in Clec16a associated pathologies. As anticipated, tofacitinib treatment significantly attenuated the fat and weight loss and improved the survival of Clec16a KO mice (FIGS. 10-A and B). Tofacitinib-treated control mice stayed healthy and maintained their body weight throughout the study.

To address the underlying mechanism behind the tofacitinib effect, we first performed RT-PCR for SOCS1 and SOCS3 expression and immunoblot analysis on gWAT isolated from controls, Clec16a KO and Clec16a KO tofacitinib-treated mice. We evaluated p-HSL, p-STAT1, p-STAT3, SOCS-1, AMPK, mTOR, P62 and LC3I/II and ER stress (FIG. 10 C-F). SOCS1 mRNA expression from gWAT of tofacitnib-treated KO mice shows significant reversal in comparison to Clec16a KO mice (FIG. 10G). SOCS3 expression showed no significant difference between the groups (FIG. 10H).

Immunoblot analysis revealed significant upregulation of phospho-HSL in Clec16a KO and reduction in the tofacitinib-treated Cle16a KO mice. Examination of adipose tissue in Clec16a KO mice demonstrated upregulation of p-STAT1 and p-STAT3. Tofacitinib rescued the inflammatory phenotype by downregulating both p-STAT1 and p-STAT3 to control levels. We also observed a significant increase in phosphorylation of AMPK. However, its target, ACC, exhibited reduced phosphorylation in the Clec16a KO mice. Tofacitnib treatment significantly reduced the p-AMPK and promoted phosphorylation of ACC. Another downstream effector of AMPK is mTOR signaling that regulates a plethora of functions, including autophagy. Over-activation of mTOR promotes inhibition of autophagy/lipophagy as evident by significant accumulation of P62 in gWAT of Clec16a KO mice. We observed a significant increase in phosphorylation of mTOR and reversal in tofacitinib treated Clec16a KO mice, correcting the autophagy defect (FIG. 10-C, D). We also evaluated for lipolytic and ER stress rescue in tofacitinib-treated KO mice (FIGS. 10-E, F). As anticipated, tofacitinib-treated KO mice showed significant downregulation in p-HSL, and ER stress proteins (GRP78, ATF6, p-IRE1α, XBP1 and CHOP). Tofacitnib treatment reduced expression of COX-2 and p-IkBα significantly as well. Increased cytokines/chemokine levels reflect upon the inflammatory mechanism utilized during the development, progression and pathogenesis of various autoimmune/inflammatory diseases. Our results indicate that Clec16a knockout inflammatory phenotype is attenuated by Tofacitinib. Quantitation of cytokines and chemokines in Clec16a KO plasma showed robust upregulation of IFN-γ, IL-1a, IL-3, IL-6, IL-13, IL-16, TNF-α, several monocyte/macrophages chemoattractant proteins and IL-17 in comparison to control. We observed near complete reversal of the inflammatory cytokines and chemokines in the tofacitinib-treated mice compared to Clec16a KO alone (FIG. 10-I).

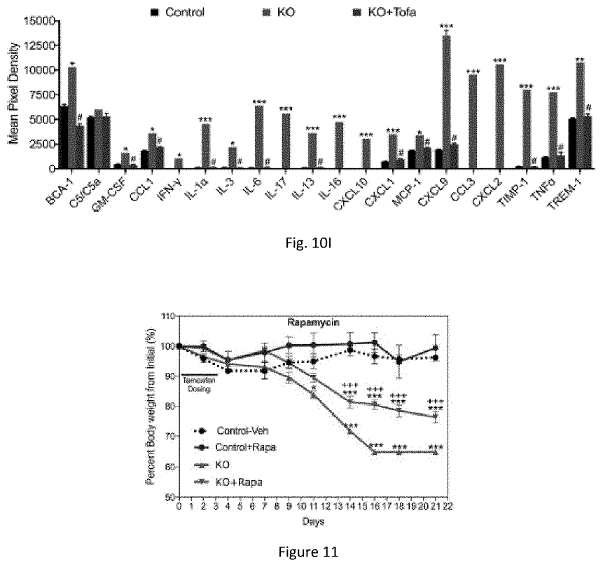

Taken together, tofacitinib exerts its multifaceted effect on HSL-mediated lipolysis, AMPK, mTOR, JAK-STATs, and autophagy/lipophagy and ER stress signaling, improves survival, and attenuates the inflammatory lipodystrophic phenotype exhibited by Clec16a KO mice. mTOR and AMPK are the core energy sensors and master regulators of cellular homeostasis. Rapamycin inhibits mTOR signaling stimulating mitophagy/autophagy through AMPK and ULK1 activation. In light of elevated ER stress and dysregulated autophagy in adipose tissue of Clec16a KO mice, we evaluated phenotype rescue of Clec16a KO with rapamycin. As anticipated, rapamycin treatment significantly reduced severe weight loss and improved the survival of Clec16a KO mice similar to tofacitinib (FIG. 11) possibly by modulating autophagy, ER stress and downstream activators of lipolytic cascade.

### Clec16a KO Induces Susceptibility to Autoimmunity in Mice.

We used our inducible KO strain to test the hypothesis that altered Clec16a expression can induce autoimmune responses in a genetic background that does not spontaneously express an autoimmune phenotype (Hudson et al., 2003). This model can therefore be used not only to trace the pathogenesis of the autoimmune responses, but also to explore how Clec16a KO might trigger the autoimmune response through modified immune regulation.

### Clec16a KO-Induced Autoantibodies.

Serum samples from control and Clec16a KO mice were assayed for antibodies to various nuclear antigens using a line assay Western blot (FIG. 12). ANA-9-Line Immunoblot assay is a membrane-based enzyme immunoassay for the semi-quantitative measurement of IgG class autoantibodies to extractable nuclear antigens SS-A 52, SS-A 60, SS-B, RNP/Sm, Sm, centromere B, Jo-1, Scl-70 and ribosomal P proteins in serum or plasma. These results show that Clec16a KO led to production of antinuclear antibodies indicative of systemic autoimmune disease.

Our finding of upregulated specific antibodies in the sera from Clec16a KO mice is noteworthy as these antibodies are also found in SLE and other systemic autoimmune diseases. Further characterization of the specific target autoantibodies in this model is needed, as this may provide clues regarding the mechanisms of lost tolerance to self-antigens.

### Serum Immunoglobulin Isotyping.

Next, we determined whether loss of Clec16a results in changes in serum immunoglobulin (Ig) isotypes. Corresponding IgG subclasses were also measured in both Clec16a KO mice and control mice sera. We compared KO mice with ≤10% and ≥20% body weight loss to controls (FIG. 13). Clec16a KO mice with ≤10% body weight loss depicted significant changes in IgM, IgA and IgG subclass IgG1 and IgG2c levels (top), whereas Clec16a KO mice with ≥20% body weight loss depicted significant upregulation in IgM, IgA, IgG2b and IgG3 IgG subclasses (bottom).

IgG1 and IgG2c showed significant increase in mice at early stages of weight loss and were not significant in Clec16a KO mice ˜20% body weight loss. IgM and IgA showed significant upregulation for both weight loss categories (FIG. 13). These upregulated serum IgG isotyping results are indicative of excessive inflammatory responses in KO mice and indicate a role for CLEC16A in autoimmunity.

### Ubiquitous Inducible Knockout of Clec16a in Mice Results in Progressive Neurodegeneration Resembling Spinocerebellar Ataxia.

Our whole body inducible Clec16a KO mice exhibits a neuronal phenotype including tremors, impaired gait, and dystonic postures that worsen over time (FIG. 14). Pathological analysis revealed that degenerating sensory axons, and Purkinje cell loss in the cerebellum account for this phenotype. Activated microglia and astrocytes were found in affected regions of the CNS. Affected and unaffected regions of the CNS and PNS showed increased levels of proteins related to mitophagy and autophagy. These findings suggest that mitophagy and/or autophagy might play a role in some kinds of spinocerebellar degeneration. The selective involvement of cerebellar and primary sensory neurons models a human disease known as spinocerebellar ataxia, which has diverse genetic causes (Huang and Verbeek, 2018).

### ER Stress and Dysregulated OXPHOS Signaling in Clec16a KO DRG and TG.

RT-PCR depicting upregulated ER stress markers in DG (A) and TG (B) at day 10 and day 22 in KO mice. (C) Representative immunoblot depicting expression of CHOP in DRG and TG lysates Day 22. (D) Quantitation graph depicting fold change in expression levels of CHOP in DRG and TG. (E) Representative immunoblot depicting mitochondrial OXPHOS respiratory complex protein levels in DRG and TG lysates of KO compared to control. A cocktail antibody comprising the following subunits of respiratory complex proteins are used: NADH dehydrogenase (ubiquinone) 1 beta subcomplex 8 (NDUFB8; complex I), succinate dehydrogenase complex, subunit B, iron sulfur (SDHB/Ip; complex II), ubiquinol-cytochrome c reductase core protein II (UQCR2; complex III), cytochrome c oxidase subunit 2 (COXII; complex IV) and ATP synthase 5A (ATP 5A, Complex V). Quantification of the levels of each of the above-mentioned subunits were shown, respectively. The data was presented as % of proteins normalized to porin levels. (F) Quantitation graph depicting fold change in expression levels of OXPHOS signaling subunits in DRG and TG. Membranes were striped and reprobed for β-actin as a loading control. Data expressed as means±SE of three independent repeats. *P<0.05, **P<0.01 ***P<0.001, #P<0.0001 (Control vs. KO).

### Clec16a Mediates its Pathogenic Effect Through ISG15.

In search of candidate protein of interest, mediating these pathogenic effects we performed CLEC16A immunoprecipitation using vMALDI-MS (Mass Spec) analysis in Clec16a overexpression system. Among top ten candidates, E3 ubiquitin ligase/Interferon-Stimulated Gene 15 (ISG15) came as candidate partner of potential interest. Clec16a overexpression YTS NK cells showed downregulation of ISG15. We anticipated if ISG15 is true candidate protein partner it should be up-regulated in KO mice. ISG15 is a ubiquitin like protein whose expression and the conjugation to targets (ISGlyation) induced by infection, Interferon-α and -γ, ischemia, DNA damage, cellular stress and aging. Interferon is one of the most important alarm molecules of the human immune system and it induces cellular defense mechanism. ISG15 also has an important role in autophagy. Observations suggest that ISGylation of proteins promotes aggregation and degradation by selective autophagy through the interaction of ISG15 with P62 and HDA6 (histone deacetylase). In addition, ISG15 is upregulated in ataxia telangiectasia cells and enhances their autophagic flux, probably to compensate for the impaired proteosomal function that is caused by their constitutive activation of ISG15.

We therefore hypothesize that ISGlyation, which is activated in stress situations, orchestrates a cellular response that arrests the cell functions by inhibiting translation and enhancing p53, triggers the degradation of endosomal and newly synthesized proteins by the autophagosome and lysosome, and signals a state of alert to induce a response by the immune system. Given that ISG15 conjugation is transient and can be reversed by specific proteases, this modification may allow the recovery of the homeostatic state once the stress has ceased. As anticipated western blot analysis in neurological tissues of control and KO mice (with score-1 and score 4 disability) showed significant upregulation of ISG15. ISG15 was not detectable in control neuronal tissues.

Recent studies from other groups on ISG15, a cellular antagonist of the ubiquitin pathway, supports the idea that mitophagy may be defective due to constitutively elevated ISG15-mediated impairment of the Ubiquitin pathway with loss of Clec16a. These findings further confirm that loss of function of Clec16a contributes to neuro-degenerative phenotype possibly through ISG15 in KO mice.

Thus, our whole-body inducible Clec16a KO model provides an excellent tool to address the mechanism by which the CLEC16A risk-associated variants may lead to autoimmune/inflammatory, lipodystrophic, neurodegenerative and spinocerebellar ataxia phenotypes. Our results from the JAK pan-inhibitor-(tofacitinib) and the selective autophagy inducer, rapamycin, suggest that in patients harboring variants that result in CLEC16A hypofunction, drugs with modulatory effects on ER stress, mitophagy/autophagy/SOCS1-JAK-STAT signaling could compensate for the attenuated CLEC16A activity and present formidable candidates for targeted interventions.

### Example II

In the previous examples, we show that loss of CLEC16A leads to abnormal mitophagy, cell death and immune dysfunction. To extend these observations we have performed immunohistochemistry (IHC) studies, including 14 formalin-fixed paraffin-embedded (FFPE) organs and tissues from Clec16a KO and control mice to reveal any visible structural defects in response to defective mitophagy. Specifically, we have harvested and studied diaphragm, heart, liver, kidney, lung, pancreas, spleen, skin, thymus, quadriceps muscle, salivary glands, triceps muscle, paw joints and intercostal muscle (N=4 per group). We show dramatic pathological differences in spleen, thymus and pancreas. Specifically, significant thymus and spleen atrophy as well as degeneration and immune cell infiltration of pancreas in Clec16a KO versus controls mice.

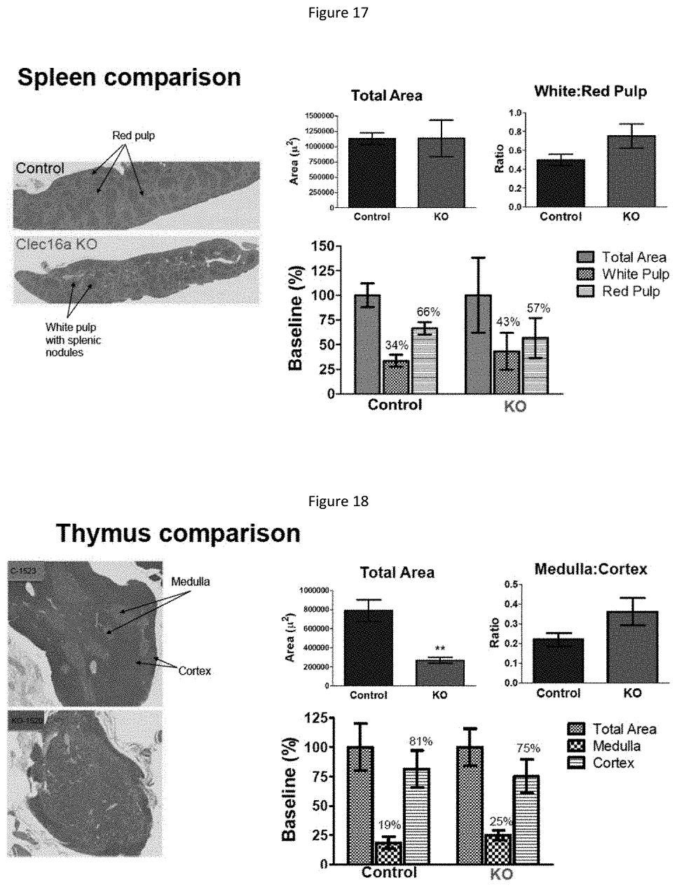

Representative images and quantification analysis for spleen are shown in FIGS. 17A and 17B. IHC revealed drastic changes in splenic architecture; red and white pulp is not well defined in KOs; white pulp predominates 43% in KOs versus 23% in controls (White:Red pulp ratios are 0.52 and 0.75 in controls and KO, respectively). It is clear that Clec16a KO mice spleen exhibit atrophy. KO mice spleen are reduced in size with predominant red pulp area in comparison to control.

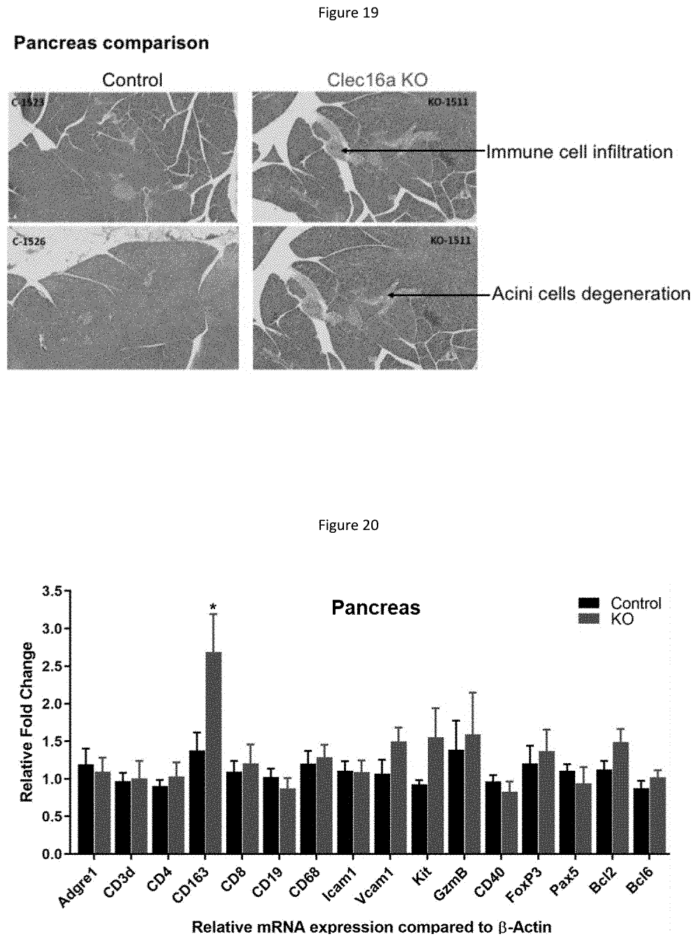

Representative images and quantification analysis for thymus are shown in FIGS. 18A and 18B. IHC revealed drastic changes in thymic architecture; thymic medullary and cortical area are not as well defined in KO mice; and medullary area is increased by 30% in KOs when compared to control (Medulla:Cortex ratios are 0.23 and 0.33 in controls and KO, respectively). Like the atrophy observed in the spleen, Clec16a KO mice thymus exhibit atrophy. KO mice thymus lobe is generally smaller in size with predominant medullary area. FIG. 19 shows pancreas of all Clec16a KO mice had significant degeneration and immune cell infiltration, not observed in pancreas of control mice.

To determine predominant type(s) of pancreas infiltrating immune cells and state of their activation we performed immunophenotyping of 16 immune markers in pancreas at Day 23 post-induction of Clec16a KO. See FIG. 20. At this time point, we only observed significant upregulation of CD163. The hemoglobin (Hb) scavenger receptor, CD163, is a macrophage-specific protein. High CD163 expression in macrophages is a characteristic of tissues responding to inflammation in a number of diseases (and in particular, inflammatory diseases) with increased macrophage activity.

Our findings are in keeping with CLEC16A being a well-documented type 1 diabetes (T1D) susceptibility gene and provides additional support for the direct involvement of CLEC16A in regulation pancreas immune cell profile.

In addition, considering the fast evolving symptoms after Clec16a KO induction, we performed a time course expression study of the 16 immune markers, as depicted above, in spleen and thymus and documented a wide dysregulation of the expression profile in Clec16a KO mice when compared to controls. To exclude effect of tamoxifen (Tam) on the expression profile we have had two control groups (Cntrl-Veh and Cntrl-Tam). There were no significant differences between two control groups. At the same time, we observed significant differences in immune markers expression between spleen and thymus. All significantly dysregulated markers are presented below.

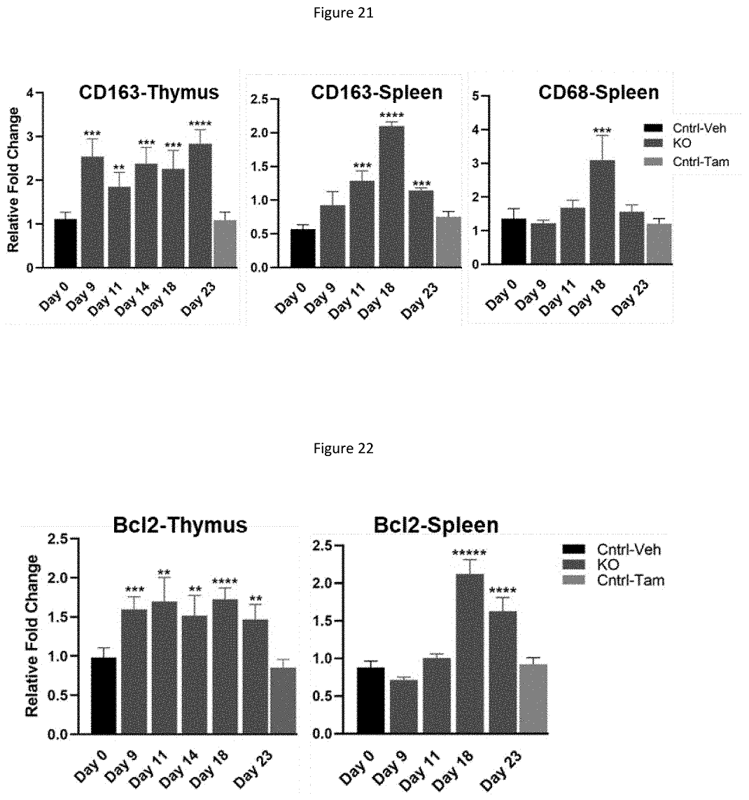

Significant upregulation of CD163 was observed and stayed stable in thymus at every studied time point and was transient increased in spleen with highest at Day 18. In addition, spleen exhibited threefold increase for CD68. Thus, we see an increase of two markers of macrophage lineage. See FIG. 21.

Significant upregulation of Bcl-2 was observed and stayed stable in thymus at every studied time point and reached a very significant increase in spleen at Day 18 and stayed up till the end of study. (FIG. 22). BCL2 is localized to the outer membrane of mitochondria, where it plays an important role in promoting cellular survival and inhibiting the actions of pro-apoptotic proteins. Apoptosis plays an active role in regulating the immune system and defective apoptosis may contribute to etiological aspects of autoimmune diseases. In addition, BCL2 is known to regulate mitochondrial dynamics, and is involved in the regulation of mitochondrial fusion and fission.

CD40 is significantly upregulated in spleen of Clec16a KOsCD40 is a costimulatory protein found on antigen-presenting cells and is required for their activation. The protein receptor encoded by this gene is a member of the TNF-receptor superfamily. This receptor is essential in mediating a broad variety of immune and inflammatory responses including T cell-dependent immunoglobulin class switching and memory B cell development.

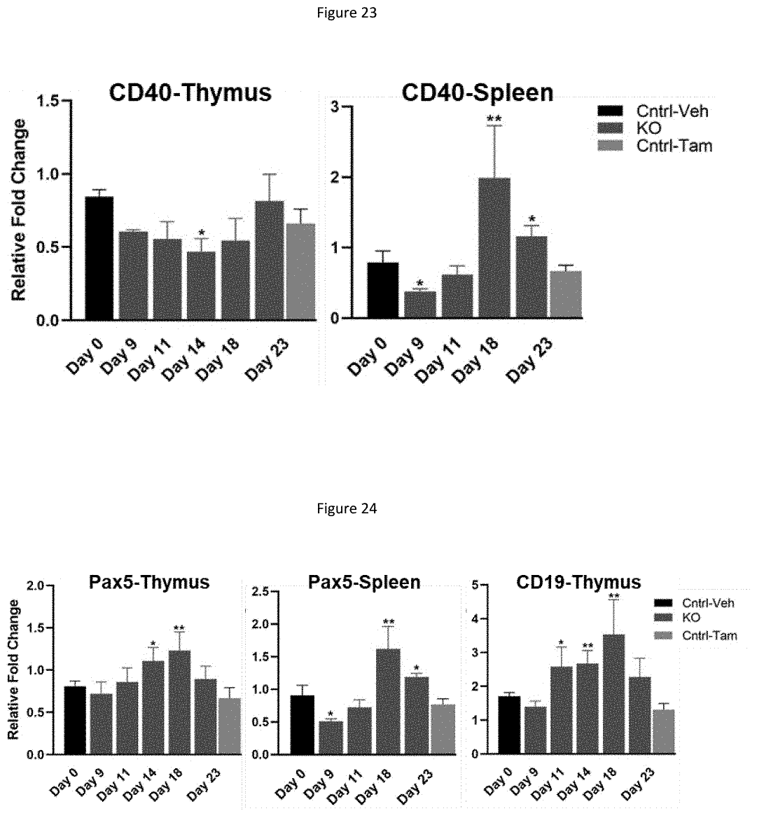

We documented upregulation of B-cell lineage markers in both, spleen and thymus from Clec16a KO mice. See FIGS. 23 and 24. CD19 is expressed in all B lineage cells. The PAX5 gene is a member of the paired box (PAX) family of transcription factors. The PAX5 gene encodes the B-cell lineage specific activator protein (B SAP) that is expressed at early, but not late stages of B-cell differentiation. Its expression has also been detected in developing CNS and testis, therefore, PAX5 gene product may not only play an important role in B-cell differentiation, but also in neural development and spermatogenesis.

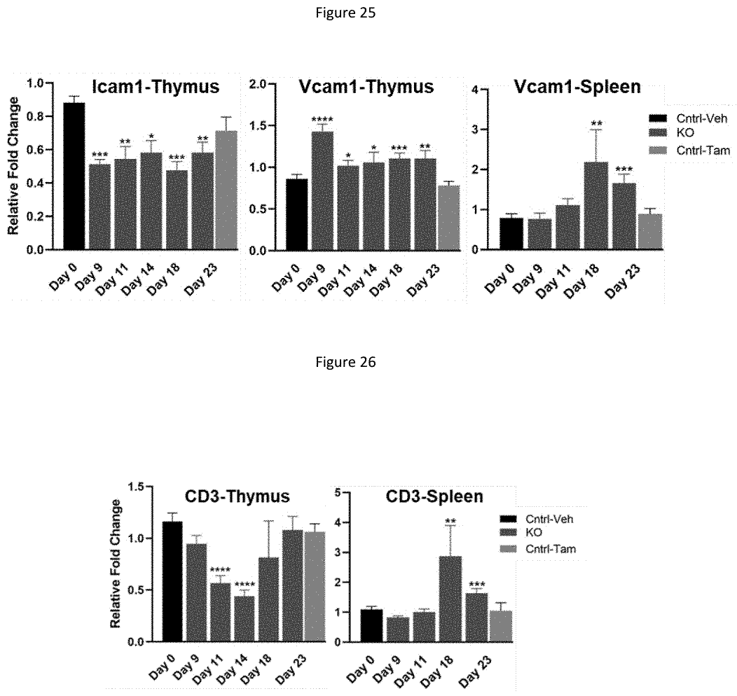

We discovered significant downregulation of Icam1 in thymus only (FIG. 25). ICAM1 is a member of the immunoglobulin superfamily, the superfamily of proteins including antibodies and T-cell receptors. ICAM1 (Intercellular Adhesion Molecule 1) also known as CD54 is a protein encoded by the ICAM1 gene. ICAM1 is an endothelial- and leukocyte-associated transmembrane protein known for its importance in stabilizing cell-cell interactions and facilitating leukocyte endothelial transmigration. ICAM1 ligation produces proinflammatory effects such as inflammatory leukocyte recruitment by signaling through cascades involving a number of kinases, thus reduction in ICAM1 could increase inflammation and modify signal transduction.

Significant upregulation of Vcam1 was observed and stayed stable in thymus at every studied time point (the highest and most significant upregulation recorder at Day 9) and was transiently increased in spleen with highest at Day 18. Vascular cell adhesion molecule 1 (VCAM-1) or CD106 is a protein encoded by the VCAM1 gene. VCAM1 functions as a cell adhesion molecule. The gene product is a cell surface sialoglycoprotein, a type I membrane protein that is a member of the Ig superfamily. The VCAM1 protein mediates the adhesion of lymphocytes, monocytes, eosinophils, and basophils to vascular endothelium. It also functions in leukocyte-endothelial cell signal transduction, and it may play a role in the development of atherosclerosis and rheumatoid arthritis. Upregulation of VCAM-1 in endothelial cells by cytokines occurs as a result of increased gene transcription.

The most extensive dysregulation of expression was observed for T cell immune markers. Expression of CD3 was significantly dysregulated both in thymus and spleen, with opposite effect. CD3 is a protein complex and T cell co-receptor that is involved in activating both the cytotoxic T cell (CD8+ naive T cells) and T helper cells (CD4+ naive T cells). See FIG. 26. CD4 was significantly down regulated in thymus, with a trend to be upregulated in spleen at Day 18, which did not reach significance (p=0.06 and p=0.09 for Day 18 and Day 23, respectively).

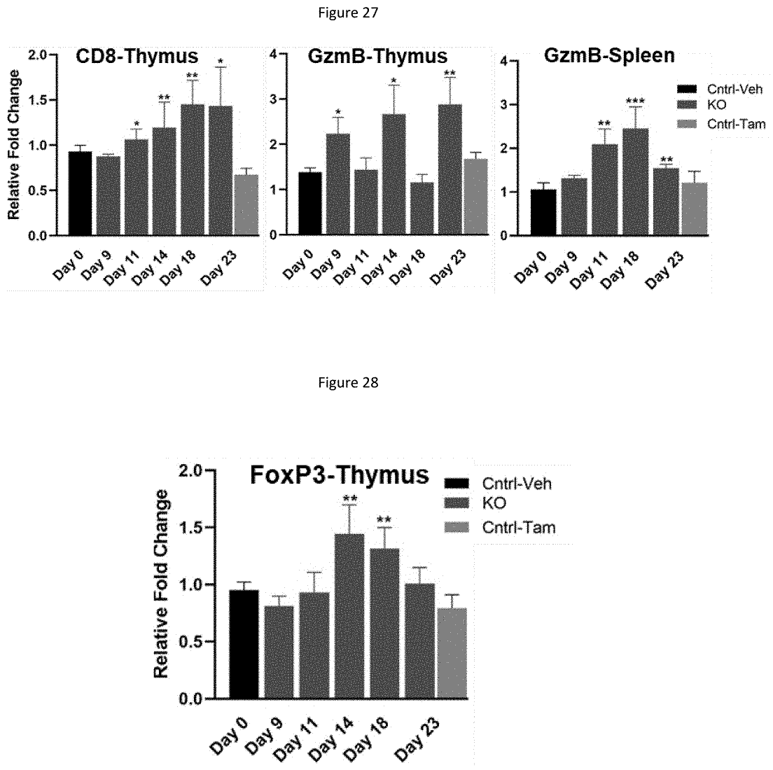

CD8 was significantly down regulated in thymus only. See FIG. 27. CD8 is a transmembrane glycoprotein that serves as a co-receptor for the T-cell receptor (TCR). The CD8 is predominantly expressed on the surface of cytotoxic T cells, but can also be found on natural killer cells, cortical thymocytes, and dendritic cells. Along with the TCR, the CD8 plays a role in T cell signaling and aiding with cytotoxic T cell antigen interactions.

Granzyme B is a serine protease encoded by the GZMB gene and expressed by cytotoxic T lymphocytes (CTL) and natural killer (NK) cells. This protein is crucial for the rapid induction of target cell apoptosis by CTL in cell-mediated immune response.

FoxP3 was transiently upregulated in thymus of Clec16a KO mice. FOXP3 (forkhead box P3) is a protein involved in immune system responses. FOXP3 is a master regulator of the regulatory pathway in the development and function of regulatory T cells (Tregs). Tregs generally turn the immune response down. The upregulation of FoxP3 could be an initial compensatory mechanism, which fails by Day 23.

### Conclusion

Our results underscore critical role of CLEC16A action in immune cells and indicate that a delicate balance of CLEC16A activity appears to be needed for cellular homeostasis. In our study, we discovered that turning off Clec16a in 8-10-week-old mice leads to robust inflammatory response, development of severe neurological symptoms, including ataxia with progressive neurodegeneration resembling spinocerebellar ataxia and significant weight loss. In, patient populations harboring variants that result in CLEC16A hypofunction, drugs with modulatory effects on mitophagy/autophagy/SOCS1/ISG15 signaling could compensate for the attenuated CLEC16A activity and present formidable candidates for targeted interventions in autoimmune and autoinflammatory diseases. Our results provide additional support for the direct involvement of CLEC16A in regulation pancreas, spleen and thymus functions.

Identification of these dysregulated gene targets and cell subpopulations allow us to choose drugs for reversing the abnormal processes in Clec16a KO mice that should have efficacy for treating and preventing symptoms of autoimmune disorders, such as T1D, in individuals with risk associated CLEC16A variants.

### REFERENCES

Fenner, J. E., Starr, R., Cornish, A. L., Zhang, J. G., Metcalf, D., Schreiber, R. D., Sheehan, K., Hilton, D. J., Alexander, W. S., and Hertzog, P. J. (2006). Suppressor of cytokine signaling 1 regulates the immune response to infection by a unique inhibition of type I interferon activity. Nat Immunol 7, 33-39.
+Khaibullin, T., Ivanova, V., Martynova, E., Cherepnev, G., Khabirov, F., Granatov, E., Rizvanov, A., and Khaiboullina, S. (2017). Elevated Levels of Proinflammatory Cytokines in Cerebrospinal Fluid of Multiple Sclerosis Patients. Front Immunol 8, 531.
Leikfoss, I. S., Mero, I. L., Dahle, M. K., Lie, B. A., Harbo, H. F., Spurkland, A., and Berge, T. (2013). Multiple sclerosis-associated single-nucleotide polymorphisms in CLEC16A correlate with reduced SOCS1 and DEXI expression in the thymus. Genes Immun 14, 62-66.
Redmann, V., Lamb, C. A., Hwang, S., Orchard, R. C., Kim, S., Razi, M., Milam, A., Park, S., Yokoyama, C. C., Kambal, A., et al. (2016). Clec16a is Critical for Autolysosome Function and Purkinje Cell Survival. Sci Rep 6, 23326.
Soleimanpour, S. A., Gupta, A., Bakay, M., Ferrari, A. M., Groff, D. N., Fadista, J., Spruce, L. A., Kushner, J. A., Groop, L., Seeholzer, S. H., et al. (2014). The diabetes susceptibility gene Clec16a regulates mitophagy. Cell 157, 1577-1590.
Tam, R. C., Li, M. W., Gao, Y. P., Pang, Y. T., Yan, S., Ge, W., Lau, C. S., and Chan, V. S. (2017). Human CLEC16A regulates autophagy through modulating mTOR activity. Exp Cell Res 352, 304-312.
Huang M, Verbeek DS. (2018). Why do so many genetic insults lead to Purkinje Cell degeneration and spinocerebellar ataxia? Neurosci Lett. 2018 Feb. 5. pii: S0304-3940(18)30080-6.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method for treating a CLEC16A-associated autoimmune disorder in a subject in need thereof, comprising administration of an effective amount of one or more agents selected from a mitophagy suppressor/modulator, an ER suppressor, a JAK2 inhibitor and SOCS1 inhibitor.

2. A method for treating CLEC16A-associated degeneration of the thymus, comprising administration of an agent that modulates expression of one or more of CD163, Bcl-2, Pax-5, V-cam1, CD8 and FoxP3, thereby altering the medulla cortex ratio in thymus and ameliorating symptoms associated with degeneration of the thymus.

3. A method for treating CLEC16A-associated degeneration of spleen comprising administration of an agent that modulates one or more of CD163, CD68, Bch 2, CD40, Pax5, Vcam1, CD3, and GzmB, thereby altering the white red pulp ratio in the spleen and ameliorating symptoms associated with degeneration of the spleen.

4. A method for treating CLEC16A-associated degeneration of the pancreas comprising administration of an agent which modulates CD163 expression and/or immune cell infiltration and/or acini cell degeneration in the pancreas, thereby reducing autoimmune symptoms.

5. The method of claim 1, wherein said CLEC16A associated autoimmune disorder is selected from type-1 diabetes, multiple sclerosis, primary adrenal insufficiency, Crohn's disease, primary biliary cirrhosis, juvenile idiopathic arthritis, rheumatoid arthritis, and alopecia areata, uveitis, neurodegeneration and lupus.

6. The method of claim 1, wherein said agents are rapamycin and tofacitinib.

7. The method of claim 1, wherein a nucleic acid from said subject is first assessed for an alteration in a CLEC16a encoding nucleic acid.

8. The method of claim 2, wherein said CLEC16A associated autoimmune disorder is selected from type-1 diabetes, multiple sclerosis, primary adrenal insufficiency, Crohn's disease, primary biliary cirrhosis, juvenile idiopathic arthritis, rheumatoid arthritis, and alopecia areata, uveitis, neurodegeneration and lupus.

9. The method of claim 2, wherein said agents are rapamycin and tofacitinib.

10. The method of claim 2, wherein a nucleic acid from said subject is first assessed for an alteration in a CLEC16a encoding nucleic acid.

11. The method of claim 3, wherein said CLEC16A associated autoimmune disorder is selected from type-1 diabetes, multiple sclerosis, primary adrenal insufficiency, Crohn's disease, primary biliary cirrhosis, juvenile idiopathic arthritis, rheumatoid arthritis, and alopecia areata, uveitis, neurodegeneration and lupus.

12. The method of claim 3, wherein said agents are rapamycin and tofacitinib.

13. The method of claim 3, wherein a nucleic acid from said subject is first assessed for an alteration in a CLEC16a encoding nucleic acid.

14. The method of claim 4, wherein said CLEC16A associated autoimmune disorder is selected from type-1 diabetes, multiple sclerosis, primary adrenal insufficiency, Crohn's disease, primary biliary cirrhosis, juvenile idiopathic arthritis, rheumatoid arthritis, and alopecia areata, uveitis, neurodegeneration and lupus.

15. The method of claim 4, wherein said agents are rapamycin and tofacitinib.

16. The method of claim 4, wherein a nucleic acid from said subject is first assessed for an alteration in a CLEC16a encoding nucleic acid.
